(19) United States
(12) Patent Application Publication
(10) Pub. No.: US 2023/0397971 A1
(43) Pub. Date: Dec. 14, 2023

(54) LATENCY FREE REMOTE PROCEDURE SUPPORT PLATFORM, PROCESSES AND PRODUCTS THEREBY

(71) Applicant: Harrison Bay Life Sciences, LLC, Harrison, TN (US)

(72) Inventors: Ron Pirucki, Coto de Caza, CA (US); Steve Redenbaugh, Harrison, TN (US); Oliver Noval, Rancho Santa Margarita, CA (US); Daniel Schulze, Keller, TX (US); Marat Yanavitski, Woodbury, MN (US); Mudassar Ahmed, Edina, MN (US)

(21) Appl. No.: 18/332,094

(22) Filed: Jun. 9, 2023

Related U.S. Application Data
(60) Provisional application No. 63/351,374, filed on Jun. 11, 2022.

(51) Int. Cl. A61B 90/00; G16H 40/67

(52) U.S. Cl. CPC A61B 90/37; A61B 90/361; G16H 40/67; A61B 2090/365; A61B 2560/0431

(57) ABSTRACT

The present inventions provide a portable, reliable open and go system for training, proctoring and procedure support during medical interventions, procedures and all manner of diagnostics extending access to care to those unable to reach other facilities, among other things. The Envisimed™ brand of system brings near zero latency Cross Reality (X-Reality/XR) technology to medical procedure support and remote collaboration, extending the reach of global health-care equity past the institutional settings that are not available to all.

### CROSS REFERENCE TO RELATED APPLICATIONS

The present filing claims the full priority benefit of U.S. provisional patent application Ser. No. 63/351,374 filed Jun. 11, 2022, and full Paris Convention Priority is claimed.

The present inventions relate to providing procedure support during surgery, telemedicine, remote proctorship and emergency/mobile medicine. The Envisimed™ brand of system brings zero latency Simultaneous or Cross Reality (X-Reality/XR) technology to procedure support and remote collaboration, along with training by allowing real time transmission and manipulation of data related to medical diagnosis, therapeutics, and HIPAA compliant procedures. Likewise, collaboration tools superseded by the instant teachings consist of traditional cloud-based medicine, prior attempts at gamification and telemedicine to date.

It is respectfully proposed that no extant systems allow for actual intervention and collaboration effective and needed at detail levels and latency-free, prior to the present invention, which highlights some of the ways the system works, without limiting this disclosure.

### BACKGROUND OF THE INVENTIONS

The Envisimed™ brand of system is a one-of-a-kind zero latency remote procedural support platform that enables users to interact and collaborate anywhere in the world, without the delays that are often experienced with traditional browser-based collaboration tools.

The Envisimed™ system utilizes a 360° camera in the procedure room via internet, cellular and/or satellite communication. The HIPAA compliant platform, featuring military grade encryption, enables remote collaborators to interact, in real-time, over the internet with a laptop, or a VR system for a more immersive interface. The remote user has control of the 360° view of the room and maneuverability for better visualization without disrupting the hospital staff during the procedure.

The ability for the remote user to point and specifically identify something in the room and communicate with minimal transmission delays experienced with traditional collaboration tools allows for a more natural interaction that enables the remote user to provide the quality of support that high acuity medical device procedures require. Connectivity is possible from anywhere in the world, complying with any safety measures in place, such as restricted hospital access, during the pandemic.

The Envisimed™ system has been purpose-built to enable medical professionals to collaborate remotely with their peers and industry partners. Its potential uses include medical device procedure support, clinical trial collaboration and support, procedural collaboration and support, training programs, and pen-operative consulting to include telemedicine and Emergency Medical Services (EMS).

Various preferred embodiments are described herein with references to the drawing in which merely illustrative views are offered for consideration, whereby:

### BRIEF DESCRIPTION OF THE FIGURES

These and other features of various embodiments disclosed herein will be better understood with respect to the following description and drawings, wherein it is respectfully submitted that no reference designators are needed at this time, except as offered for consideration.

FIG. 1A shows a first of three primary usage platforms exemplary of the applications supported by the instant systems, namely, procedure support;

FIG. 1B shows proctoring, yet another exemplary and effective use of the present inventions;

FIG. 1C illustrates the training function which is likewise embraced as proprietary and within the scope of the instant teachings according to the present inventions;

FIG. 2A provides a software dashboard view according to the instant teachings;

FIG. 2B likewise shows the software dashboard view, available during each of the usage platforms, namely in this view showing a stenting procedure;

FIG. 2C also shows the software dashboard view, namely of a clinician performing an access procedure;

FIG. 2D demonstrates a screen view of vital patient data, as known to those skilled in the art;

FIG. 3A shows the STORM type of case used to house, transport and ship the instant system;

FIG. 3B is indicative and demonstrative of a typical laptop interface, according to the instant system;

FIG. 3C shows the STORM case, effective to house the instant inventions, in a first configuration;

FIG. 3D also shows the STORM case in a second or closed configuration;

FIG. 3E details systems elements used in a typical procedure support role;

FIG. 4A provides a related view of clinicians performing procedures; and

FIG. 4B completes the set of summary vies showing the system being used.

FIG. 4C shows remote access to the Comprehensive OR Set Up via laptop, wherein clinicians may participate fully in any number of procedures according to the instant inventions.

The detailed descriptions set forth below merely illustrate zero latency examples of the instant inventions, applied to certain usages.

Corresponding reference characters indicate corresponding components throughout the several views of the drawing. Skilled artisans will appreciate that elements in the figures are illustrated for simplicity and clarity, and have not necessarily been drawn to scale. For example, the dimensions of some of the elements in the figures may be exaggerated relative to other elements to help to improve understanding of various embodiments of the present invention. Also, common but well-understood elements that are useful or necessary in a commercially feasible embodiment are often not depicted in order to facilitate a less obstructed view of these various embodiments of the present invention.

### OBJECTS AND SUMMARY OF THE INVENTIONS

Briefly stated, a 360 degree camera gamifies a procedural suite/theater using HIPAA compliant data sets in real time, as the Envisimed™ brand of system for medical monitoring, diagnosis, training and intervention.

According to embodiments, there are disclosed systems for providing real-time near latency free visual and acoustic access to medical procedure rooms/suites/theaters, which comprising at least an open-and-go container with a plurality of connection ports, able to be set up within 10 minutes, housing at least a 360 degree camera set, with acoustical sensing and transmitting ability, secured connections to end users, HIPAA compliant data streams, and proprietary software running connections to special and or general purpose portable computing machines to register and compose data sets.

According to embodiments there are disclosed fully resourced medical diagnostics, therapeutics and inter-optioned equipment combined with panning and zooming views creating delimited procedural access to remote and proximate users and patients purpose-built platforms which clinicians can use to eliminate pitfalls of traditional collaboration tools.

According to embodiments, there are provided, an open and go system for managing at least one of training, proctoring and procedure support, further comprising, in combination:
at least a container for housing a plurality of omnidirectional cameras and acoustical sensors and transmitters;
customizable data streams for registering and composing images for medical device-based diagnosis, demonstration and procedure support; and,
remote connectivity without latency, whereby real-time medical, interventional and procedural support options may be arrayed, sampled and combined to support optimized outcomes; an Alienware Gaming Computer;

### an Axis PTZ Camera;

### a Router; and

ENVISIMED type of Software for registering and composing visual and acoustical images and waveforms.

According to embodiments, there is provided, an improved telemedicine platform being fully gamified, portable and able to be rapidly deployed, which comprises, in combination:
a fully equipped procedure room having real-time connections to relevant medical imaging and theatre specific omnidirectional three-hundred and sixty degree views;
HIPAA compliant patient data via proprietary software;
remote clinician and physician access, and,
   registered, synchronized and composed aggregates of medical device signals used to compose salient data sets, being software which coordinates lag-free comprehensive access to all patient, provider and equipment driven information, mediated by humans, and supervised by gated AI.

### DETAILED DESCRIPTIONS

The present inventors have discovered that all of the benefits and medical support needed for procedural support, proctoring and training can be provided with an open and go system housed in readily portable and shippable containers. With under 10 minutes and a plug and play approach, the instant systems extend the reach of global healthcare equity to those not having access to care, along with proctoring and training without latency to interrupt the integrity Incorporated by reference as if fully set forth herein, in their entirety, are the following U.S. Pat. Nos. 5,473,397; 5,497,150; 5,527,714; 5,562,012; 5,972,019; 5,980,595; 6,010,498; 6,051,020; 6,306,141; 6,418,301; 7,008,403; 7,620,523; 7,467,067; 7,705,285; 8,066,757; 8,088,140; 8,545,514; 8,585,713; 8,926,680; 8,945,143; 8,945,172; 9,808,359; 9,198, 687; 9,220,522; 10,016,638; 10,960,313; 10,964,508; 11,043,036; 11,187,486; 11,282,284; and, 11,310,368.

The present inventors transmit without latency full remote access to any case, using sophisticated medical device technology, via a fully integrated remote procedure support platform. Leveraging augmented and virtual reality platforms with proprietary software enables merger of a 360 degree omnidirectional camera and registered and integrated medical device data, imagery, and timing elements.

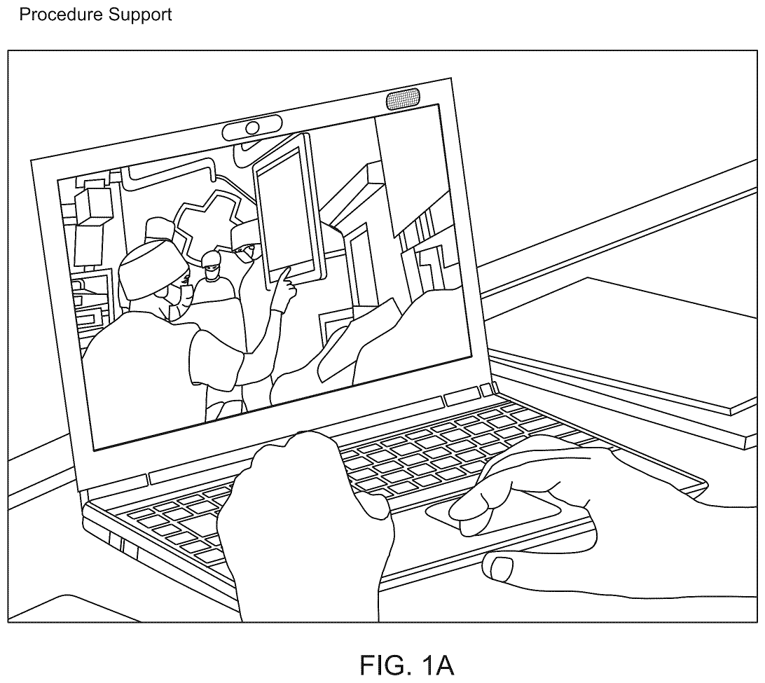

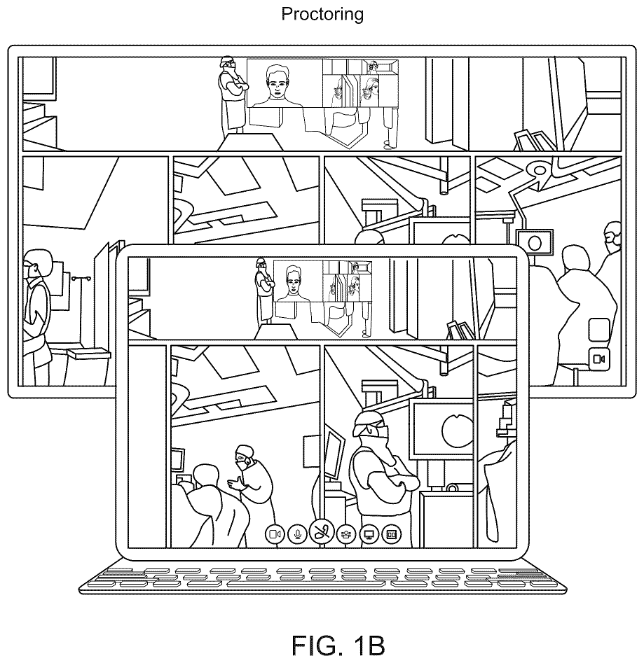

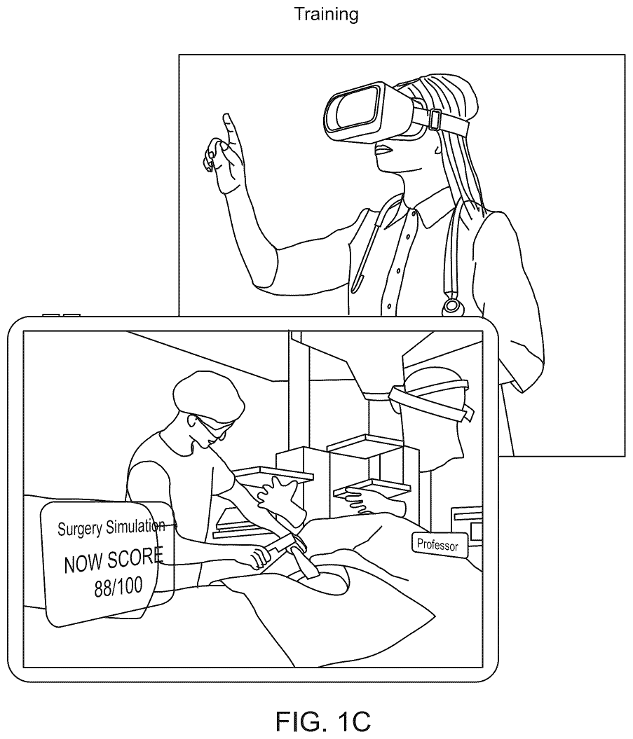

Referring now to FIG. 1A, procedure support is illustrated. Conventional Procedure Support Costs include time and money savings according to the instant systems. Time to destination, preparing for travel, time related to scheduling days, time away from family, time related to travel delays and spatial limitations are each addressed by using the instant system. Likewise the hard (money) costs of airfare/ground transportation, lodging, credentialing, hazardous exposure and liability are mitigated by the ENVISIMED brand of system.

Referring now also to FIGS. 1B, 1C prior to the instant system, the unmet needs related to case support also apply to proctoring and training, namely, there is often a criticality to commercializing sophisticated devices, cost prohibitivity to start-ups and the available remoter case support technology is often unsatisfactory. Remote procedure support means that a surgical suite with a 360 degree camera technology offers three hundred sixty degree omnidirectional view of the room. Likewise, the ENVISIMED brand of system features minimum latency, minimum footprint, a realistic interface, the versatile, easily assembled remote personnel support and logistical separation.

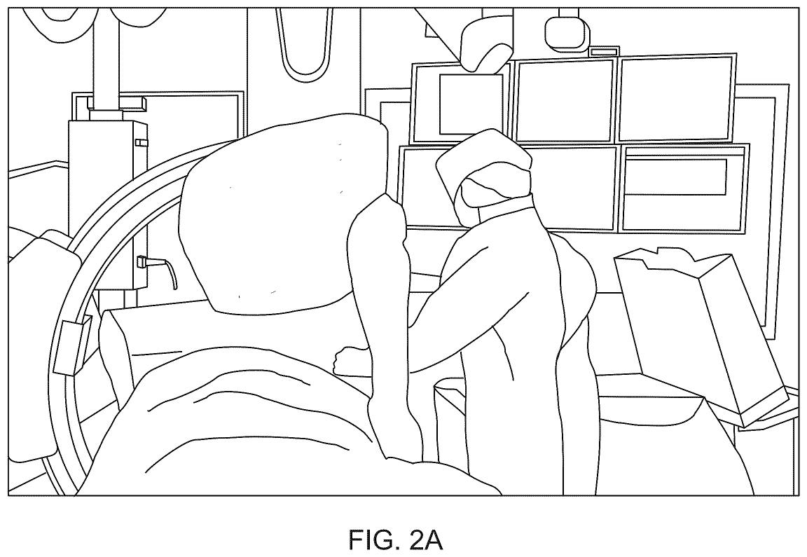

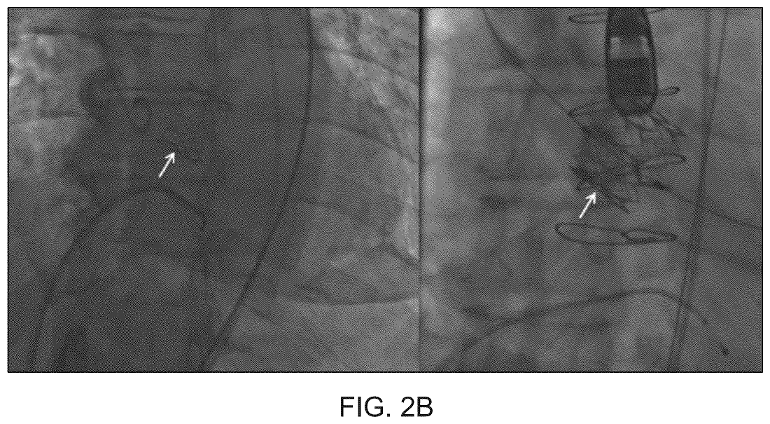

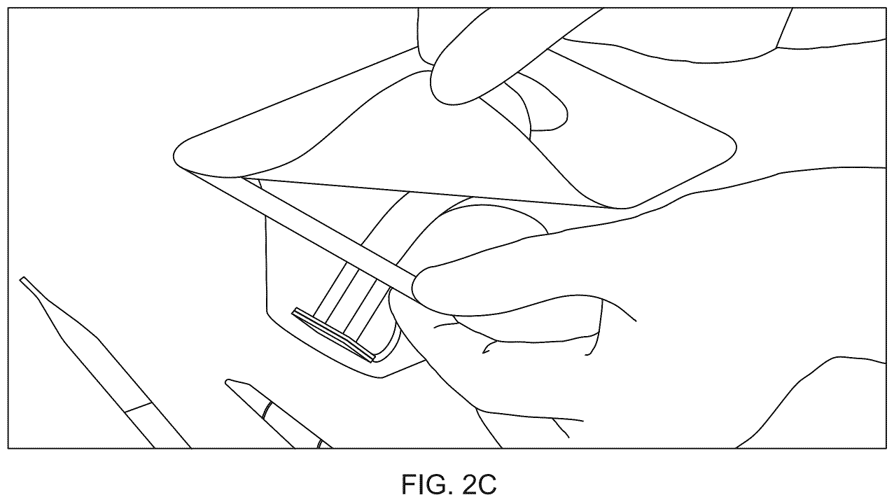

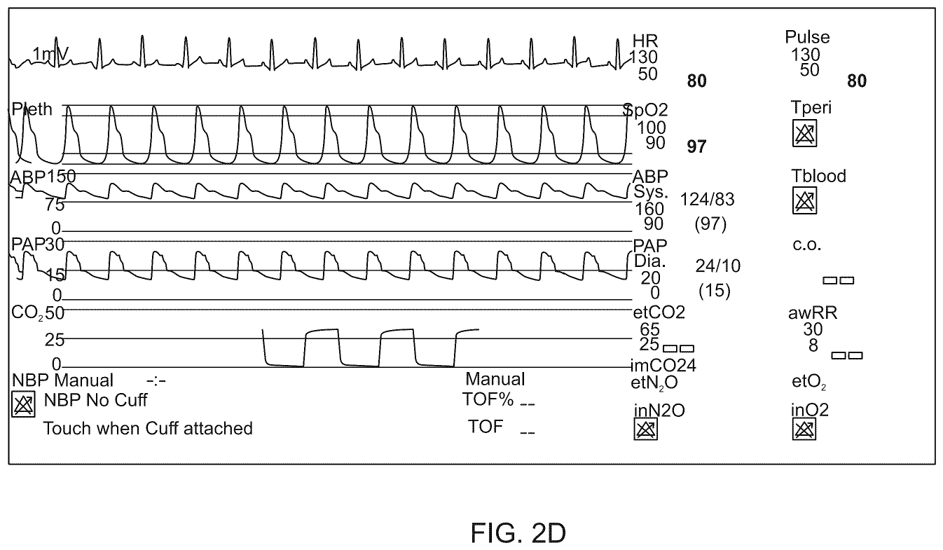

Referring now to the Software Dashboard View of FIGS. 2A, 2B and 2C it is noted that visual obstruction is prevented, for example the 2A view shows that the patient can be seen on the table with the clinician and surgical drape, arrayed next to screens showing vital signs and this supports each of the three aspects of procedure support, training and proctoring as full visual and acoustic access is provided.

FIG. 2B shows a radiographic view including a stent/flow diverter or related implant which is emplaced within a lesion being treated. The directional arrows indicate that the placement was successful and are visible to all involved. FIG. 2c shows preparation of the access site, also freely viewable and effective to teach others, proctor and confirm that all of the steps needed to be managed have been taken.

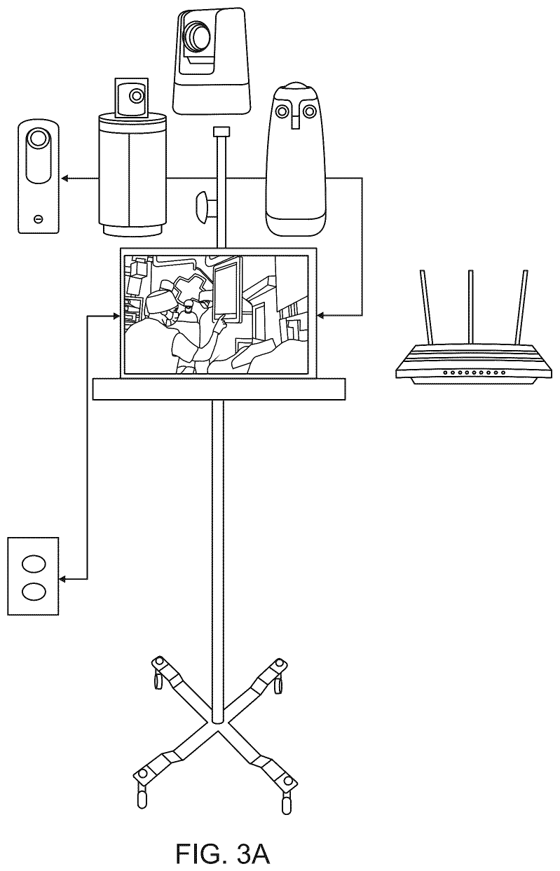

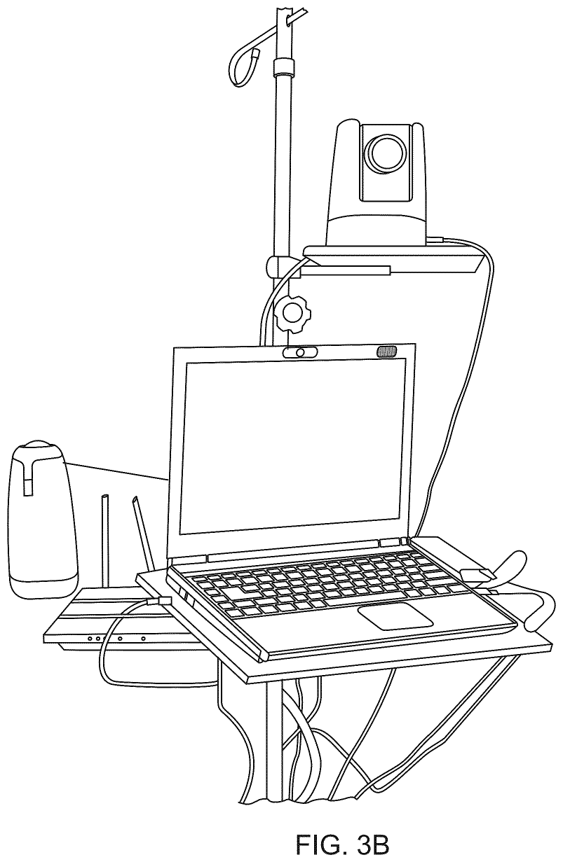

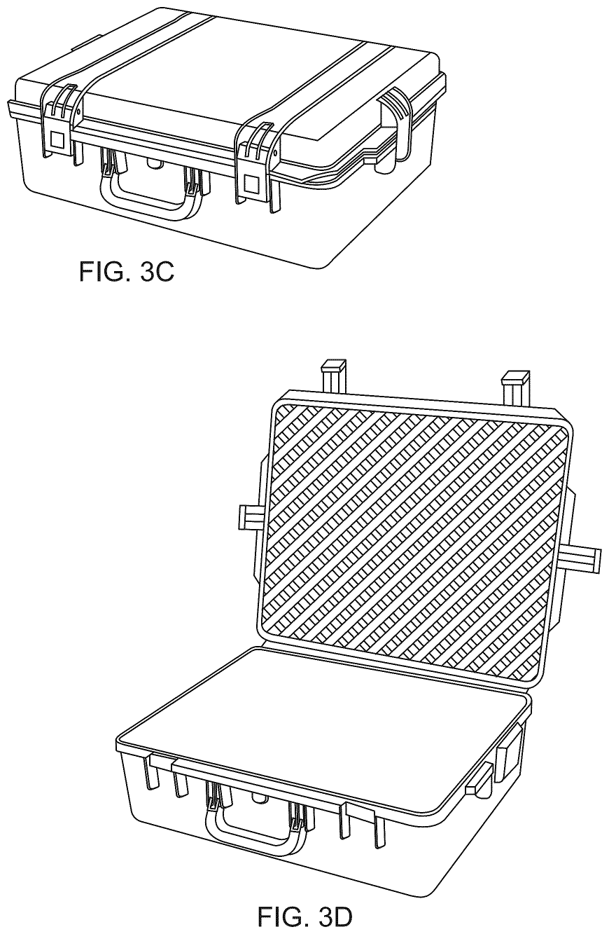

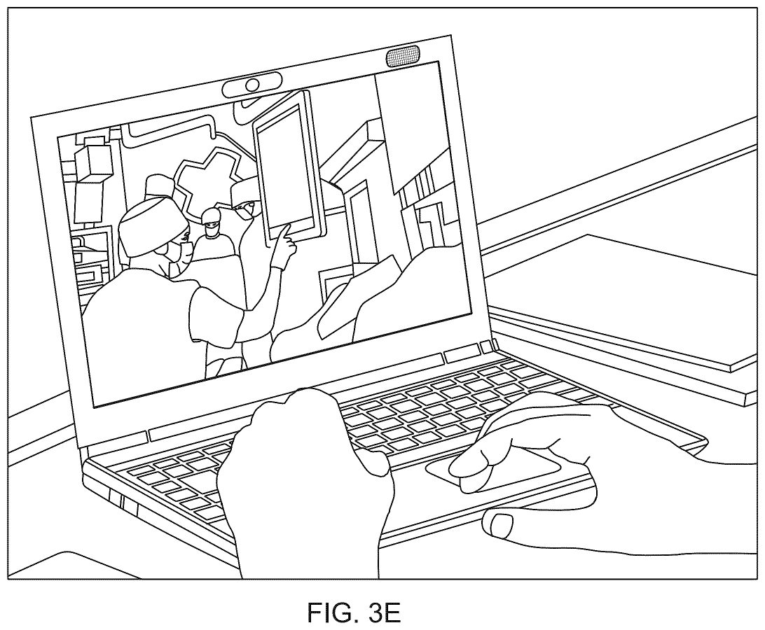

FIG. 3A shows an exemplary, but not limiting embodiment of a system of the present invention, with 360 degree cameras (Owl Labs Meeting Owl 3 Premium Pack/Kandao Meeting Meeting 360/Axis Communication V5925 1080p PTZ Network Camera), a laptop screen mounted on a platform, and acoustical apparatus serially linked and operationally connected both to a power source, and signal transmitting and transferring pick-up and router/modem-means being customizable, zero latency, reasonably priced, 256-bit data security based, internet/WiFi/HW/Cellphone and hot-spot driven and/or Satellite makes the instant system, including ViaSAT and or MARS as is known to artisans to broaden access. The illustrated system includes for example a ThinkPadX1 CarbonGen 10 Intel (14″) Black/Alienware M18 Gaming laptop which using the instant software effectively composes and registers multiple inputs to support the procedure/training/proctoring FIG. 3B likewise shows an exemplary system for procedure support, proctoring and training usages, onsite Emergency Medicine. Being (near) zero latency (as perceived by a human) with readily available hardware, a realistic interface, a 360 degree view, easy assembly and remote personnel support along with logistical separation allows each of these modalities to be fully gamified, and manage data which is electronically composed, registered and interfaced to be performed in real-time by any trained user.

FIG. 3C shows a STORM CASE® brand of container for housing, shipping and storing the instant systems. In FIG. 3C a first configuration is shown being closed, housing the system of the present invention and FIG. 3D the case is shown in a second position, wherein it contains the essential elements of the present invention, as shown and described herein, and claimed below. Those skilled in the art understand the interchangeability of the “off-of-the shelf” components featured herein\, as fully denoued above and claimed below. FIG. 3E likewise shows for example a ThinkPadX1 CarbonGen 10 Intel (14″) Black/Alienware M18 Gaming laptop which using the instant software effectively composes and registers multiple inputs to support the procedure/training/proctoring, in this case at the hands of a remote user.

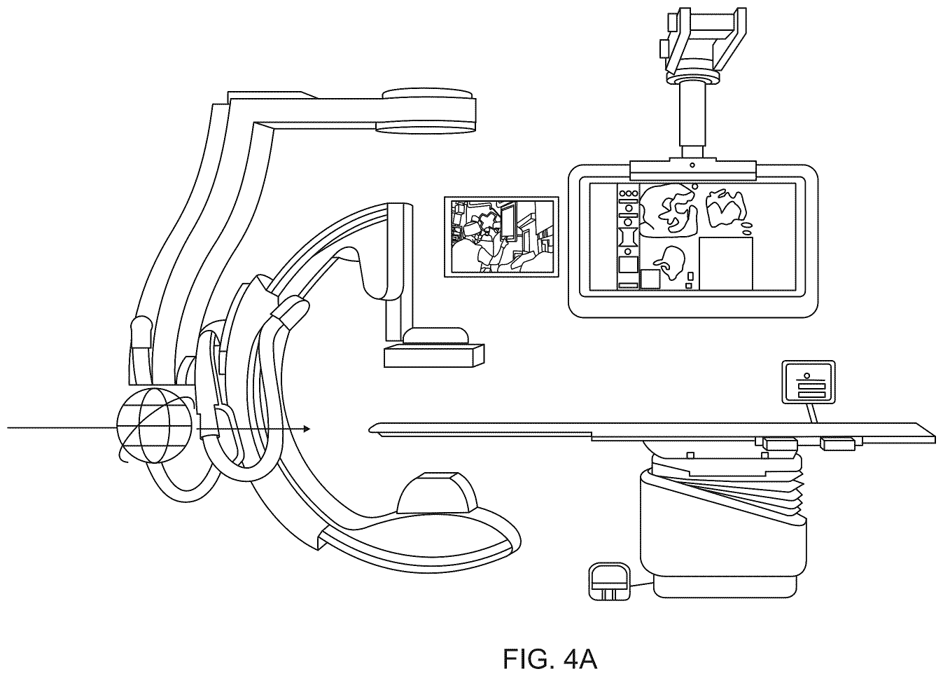

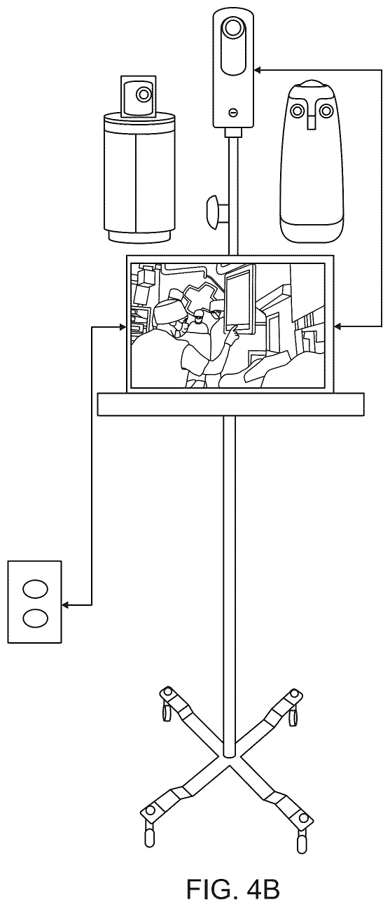

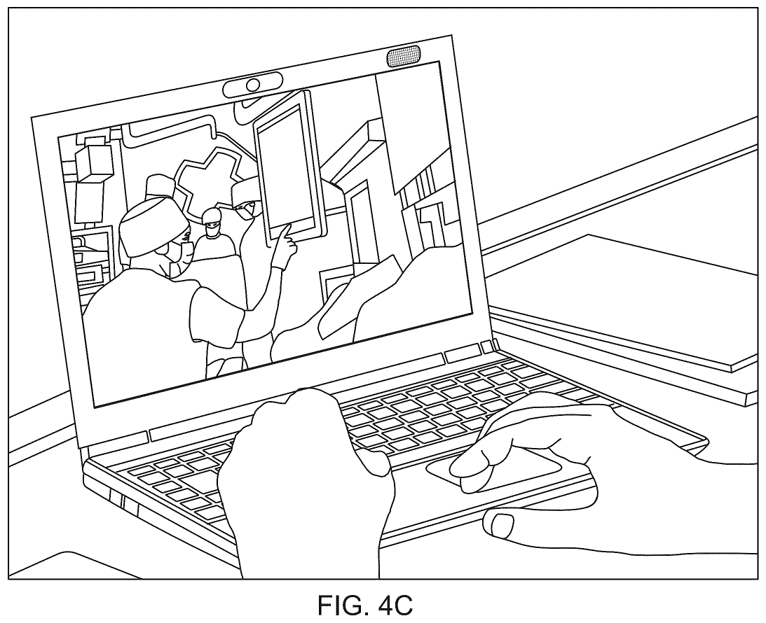

Turning now to FIGS. 4A, 4B and 4C a comprehensive Operating Room (OR) set up is offered for consideration, grounded by with 360 degree cameras (Owl Labs Meeting Owl 3 Premium Pack/Kandao Meeting Meeting 360/Axis Communication V5925 1080p PTZ Network Camera), a laptop screen mounted on a platform, and acoustical apparatus serially linked and operationally connected both to a power source, and signal transmitting and transferring pick-up and router/modem-means being customizable, zero latency, reasonably priced, 256-bit data security based, internet/WiFi/HW/Cellphone and hot-spot driven and/or Satellite makes the instant system, including ViaSAT and or MARS as is known to artisans to broaden access. The illustrated system includes for example a ThinkPadX1 CarbonGen 10 Intel (14″) Black/Alienware M18 Gaming laptop which using the instant software effectively composes and registers multiple inputs to support the procedure. As discussed throughout, the composition of registered diagnostic images interplays with off-of-the shelf components to be commanded by the software to perform a broad range of different procedures, with management of the data driving favorable and readily achievable outcomes stretching the bounds of access to treatment past known and high-cost alternatives. By way of further example, the instant system May be configured as follows; an Alienware Gaming Laptop: Alienware m15 R7 Gaming Laptop—15.6-inch 240 Hz 2 ms QHD, Intel Core i9-12900H, 32 GB DDR5 RAM, 1 TB SSD, NVIDIA Geforce RTX 3080 Graphics, Killer AX 1675i with Dell services, Windows 11 Home—Dark at least an Axis PTZ Desktop Camera: UHD 4K at 30 fps and 20× zoom, Broadcast-quality audio with XLR inputs, VISCA and VISCA over IP support, Camstreamer 3-month trial included, 3G-SDI and HDMI outputs; at least a Router: Digi EX50 5G Cellular Router. Future-proof and ready for new use cases such as 5G network slicing, SD-WAN, 4K/8K cameras and AR/VR, High-speed 5G NR and 4G LTE-Advanced Pro Cat 20 dual connectivity CBRS, C-band, FirstNet Capable™ cellular radio, Enterprise-grade Wi-Fi 6 and dual 2.5 Gigabit Ethernet, Active PoE+ switch or DC IN for primary and backup power, Extended operating temperature range for wider use-cases, Standards-based Power over Ethernet simplifies installation, Multiple mounting options and accessories, Serial port for remote, out-of-band management, Securely deploy, monitor and control devices with Digi Remote Manager®; and at least a remote Laptop: Lenovo Flex 5. Features fast 11^{th} Generation Intel® Core™ processors, Get things done efficiently on a 14 inch FHD IPS touchscreen with 360-degree hinge, Great for work, home or school boasting long battery life, Enjoy added security features like physical webcam privacy shutter & fingerprint reader, Get creative & stay on point with your tasks via optional digital pen (not included), fully registered and composed by ENVISMED type of software.

The instant system uses, for example, cutting edge medical devices (see U.S. Pat. No. 9,808,359, in Appendix) to allow practitioners to Proctor and/or Train to perform endovascular treatments, such as aneurysm coiling/stenting, while using any and all tools patented for same, such as lasers (U.S. Pat. No. 7,008,403), stent-trievers (U.S. Pat. No. 8,088,140) views offered by augmented/virtual reality (U.S. Pat. No. 10,960,038) with time-based computer communications (U.S. Pat. No. 11,310,368) along with AR/VR features such as pupillary tracking (U.S. Pat. No. 11,187,906) Artificial Intelligence (U.S. Pat. No. 11,043,036) and all associated with the instant inventions. In short, the paradigm shift of remote based telemedical procedures, and all associated needs, are supported for existing and later developed technologies by the Envisimed™ brand of system providing purpose-built platforms to perform procedure support, proctoring and training as if it were in the same room. It is respectfully proposed that this set of innovations should be subject to U.S. Letters Patents, inter alia, as set forth and claimed herein.

While several embodiments of the present disclosure have been described and illustrated herein, those of ordinary skill in the art will readily envision a variety of other means and/or structures for performing the functions and/or obtaining the results and/or one or more of the advantages described herein, and each of such variations and/or modifications is deemed to be within the scope of the present disclosure. More generally, those skilled in the art will readily appreciate that all parameters, dimensions, materials, and configurations described herein are meant to be exemplary and that the actual parameters, dimensions, materials, and/or configurations will depend upon the specific application or applications for which the teachings of the present disclosure is/are used.

Those skilled in the art will recognize, or be able to ascertain using no more than routine experimentation, many equivalents to the specific embodiments of the disclosure described herein. It is, therefore, to be understood that the foregoing embodiments are presented by way of example only and that, within the scope of the appended claims and equivalents thereto, the disclosure may be practiced otherwise than as specifically described and claimed. The present disclosure is directed to each individual feature, system, article, material, kit, and/or method described herein. In addition, any combination of two or more such features, systems, articles, materials, kits, and/or methods, if such features, systems, articles, materials, kits, and/or methods are not mutually inconsistent, is included within the scope of the present disclosure.

All definitions, as defined and used herein, should be understood to control over dictionary definitions, definitions in documents incorporated by reference, and/or ordinary meanings of the defined terms.

The indefinite articles “a” and “an,” as used herein in the specification and in the claims, unless clearly indicated to the contrary, should be understood to mean “at least one.” The phrase “and/or,” as used herein in the specification and in the claims, should be understood to mean “either or both” of the elements so conjoined, i.e., elements that are conjunctively present in some cases and disjunctively present in other cases. Other elements may optionally be present other than the elements specifically identified by the “and/or” clause, whether related or unrelated to those elements specifically identified, unless clearly indicated to the contrary.

Reference throughout this specification to “one embodiment” or “an embodiment” means that a particular feature, structure, or characteristic described in connection with the embodiment is included in at least one embodiment. Thus, appearances of the phrases “in one embodiment” or “in an embodiment” in various places throughout this specification are not necessarily all referring to the same embodiment. Furthermore, the particular features, structures, or characteristics may be combined in any suitable manner in one or more embodiments.

The terms and expressions which have been employed herein are used as terms of description and not of limitation, and there is no intention, in the use of such terms and expressions, of excluding any equivalents of the features shown and described (or portions thereof), and it is recognized that various modifications are possible within the scope of the claims. Accordingly, the claims are intended to cover all such equivalents.

Reference throughout this specification to “one embodiment,” “an embodiment,” or similar language means that a particular feature, structure, or characteristic described in connection with the embodiment is included in at least one embodiment of the present invention. Thus, appearances of the phrases “in one embodiment,” “in an embodiment,” and similar throughout this specification may, but do not necessarily, all refer to the same embodiment.

Furthermore, the described features, structures, or characteristics of the invention may be combined in any suitable manner in one or more embodiments. In the following description, numerous specific details are provided to provide a thorough understanding of embodiments of the invention. One skilled in the relevant art will recognize, however, that the invention may be practiced without one or more of the specific details, or with other methods, components, materials, and so forth. In other instances, well-known structures, materials, and so forth. In other instances, well-known structures, materials, or operations are not shown or described in detail to avoid obscuring aspects of the invention.

The schematic flow chart diagrams included herein are generally set forth as logical flow chart diagrams. As such, the depicted order and labeled steps are indicative of one embodiment of the presented method. Other steps and methods may be conceived that are equivalent in function, logic, or effect to one or more steps, or portions thereof, of the illustrated method. Additionally, the format and symbols employed are provided to explain the logical steps of the method and are understood not to limit the scope of the method. Although various arrow types and line types may be employed in the flow chart diagrams, they are understood not to limit the scope of the corresponding method. Indeed, some arrows or other connectors may be used to indicate only the logical flow of the method. For instance, an arrow may indicate a waiting or monitoring period of unspecified duration between enumerated steps of the depicted method. Additionally, the order in which a particular method occurs may or may not strictly adhere to the order of the corresponding steps shown.

Unless otherwise indicated, all numbers expressing quantities of ingredients, properties such as molecular weight, reaction conditions, and so forth used in the specification and claims are to be understood as being modified in all instances by the term “about.” Accordingly, unless indicated to the contrary, the numerical parameters set forth in the specification and attached claims are approximations that may vary depending upon the desired properties sought to be obtained by the present invention. At the very least, and not as an attempt to limit the application of the doctrine of equivalents to the scope of the claims, each numerical parameter should at least be construed in light of the number of reported significant digits and by applying ordinary rounding techniques. Notwithstanding that the numerical ranges and parameters setting forth the broad scope of the invention are approximations, the numerical values set forth in the specific examples are reported as precisely as possible. Any numerical value, however, inherently contains certain errors necessarily resulting from the standard deviation found in their respective testing measurements.

The terms “a,” “an,” “the” and similar referents used in the context of describing the invention (especially in the context of the following claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein. All methods described herein can be performed in any suitable order unless otherwise indicated herein or otherwise clearly contradicted by context. The use of any and all examples, or exemplary language (e.g., “such as”) provided herein is intended merely to better illuminate the invention and does not pose a limitation on the scope of the invention otherwise claimed. No language in the specification should be construed as indicating any non-claimed element essential to the practice of the invention.

Groupings of alternative elements or embodiments of the invention disclosed herein are not to be construed as limitations. Each group member may be referred to and claimed individually or in any combination with other members of the group or other elements found herein. It is anticipated that one or more members of a group may be included in, or deleted from, a group for reasons of convenience and/or patentability. When any such inclusion or deletion occurs, the specification is deemed to contain the group as modified thus fulfilling the written description of all Markush groups used in the appended claims.

Certain embodiments of this invention are described herein, including the best mode known to the inventors for carrying out the invention. Of course, variations on these described embodiments will become apparent to those of ordinary skill in the art upon reading the foregoing description. The inventor expects skilled artisans to employ such variations as appropriate, and the inventors intend for the invention to be practiced otherwise than specifically described herein. Accordingly, this invention includes all modifications and equivalents of the subject matter recited in the claims appended hereto as permitted by applicable law. Moreover, any combination of the above-described elements in all possible variations thereof is encompassed by the invention unless otherwise indicated herein or otherwise clearly contradicted by context.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the invention so claimed are inherently or expressly described and enabled herein.

As one skilled in the art would recognize as necessary or best-suited for performance of the methods of the invention, a computer system or machines of the invention include one or more processors (e.g., a central processing unit (CPU) a graphics processing unit (GPU) or both), a main memory and a static memory, which communicate with each other via a bus.

A processor may be provided by one or more processors including, for example, one or more of a single core or multi-core processor (e.g., AMD Phenom II X2, Intel Core Duo, AMD Phenom II X4, Intel Core i5, Intel Core I & Extreme Edition 980X, or Intel Xeon E7-2820).

An I/O mechanism may include a video display unit (e.g., a liquid crystal display (LCD) or a cathode ray tube (CRT)), an alphanumeric input device (e.g., a keyboard), a cursor control device (e.g., a mouse), a disk drive unit, a signal generation device (e.g., a speaker), an accelerometer, a microphone, a cellular radio frequency antenna, and a network interface device (e.g., a network interface card (NIC), Wi-Fi card, cellular modem, data jack, Ethernet port, modem jack, HDMI port, mini-HDMI port, USB port), touchscreen (e.g., CRT, LCD, LED, AMOLED, Super AMOLED), pointing device, trackpad, light (e.g., LED), light/image projection device, or a combination thereof.

Memory according to the invention refers to a non-transitory memory which is provided by one or more tangible devices which preferably include one or more machine-readable medium on which is stored one or more sets of instructions (e.g., software) embodying any one or more of the methodologies or functions described herein. The software may also reside, completely or at least partially, within the main memory, processor, or both during execution thereof by a computer within system, the main memory and the processor also constituting machine-readable media. The software may further be transmitted or received over a network via the network interface device.

While the machine-readable medium can in an exemplary embodiment be a single medium, the term “machine-readable medium” should be taken to include a single medium or multiple media (e.g., a centralized or distributed database, and/or associated caches and servers) that store the one or more sets of instructions. The term “machine-readable medium” shall also be taken to include any medium that is capable of storing, encoding or carrying a set of instructions for execution by the machine and that cause the machine to perform any one or more of the methodologies of the present invention. Memory may be, for example, one or more of a hard disk drive, solid state drive (SSD), an optical disc, flash memory, zip disk, tape drive, “cloud” storage location, or a combination thereof. In certain embodiments, a device of the invention includes a tangible, non-transitory computer readable medium for memory. Exemplary devices for use as memory include semiconductor memory devices, (e.g., EPROM, EEPROM, solid state drive (SSD), and flash memory devices e.g., SD, micro SD, SDXC, SDIO, SDHC cards); magnetic disks, (e.g., internal hard disks or removable disks); and optical disks (e.g., CD and DVD disks).

Furthermore, numerous references have been made to patents and printed publications throughout this specification. Each of the above-cited references and printed publications are individually incorporated herein by reference in their entirety.

In closing, it is to be understood that the embodiments of the invention disclosed herein are illustrative of the principles of the present invention. Other modifications that may be employed are within the scope of the invention. Thus, by way of example, but not of limitation, alternative configurations of the present invention may be utilized in accordance with the teachings herein. Accordingly, the present invention is not limited to that precisely as shown and described.

What is claimed is:

1. A system for providing real-time near latency-free visual and acoustic access to medical procedure rooms/suites/theaters, which comprises, in combination:
an open-and-go container with at least two connection ports, able to be set up within 10 minutes;
at least a 360 degree camera set, with acoustical sensing and transmitting ability;
secured connections to end users;
HIPAA compliant data streams;
proprietary software running connections to special and or general purpose portable computing machines to register and compose data sets; and,
fully resourced medical diagnostics, therapeutics and inter-optioned equipment combined with panning and zooming views creating delimited procedural access to remote and proximate users and patients.

2. The system of claim 1, configured to be a procedure room, interventional radiology or cardiology suite, operating theater, diagnostic and examination room;
whereby the interactive experience enables medical service providers and support personnel to review, understand, and agree upon procedures from the most highly technical to clinically routine, using conventional (“off-of-the-shelf”) tools, pointers and resources offering for consideration unobstructed visualization, recording and artificially intelligent data sets, manipulated by end-users, for real-time management of medical issues.

3. The system of claim 2, further comprising:
a set of optimized remote access tools incorporated into a system for case support, proctoring and training, as defined and shown herein, further comprising:
at least a customizable software platform;
a GPU plus platform technology;
near zero (zero human-perceived) latency;
managed pricing;
256-bit data security; and,
connectivity without issues impacting the ability to perform diagnostic, interventional and surgical procedures, consistent with the standard of care in respective medical fields.

4. The system of claim 3, effective to provide medical case support using telemedicine ported via platform, purpose-built visual and acoustic streams and using conventional technology which obviates the need to for a traditional medical suite, saving:
time to destination;
travel and preparation for same;
schedulings;
delays and spatial limitations whereby users are empowered to interact and collaborate outside of the limitation of conventional browser-based tools.

5. The system of claim 4, further comprising:
medical device procedure support, procedural collaboration and support, clinical trial collaboration and support, training programs, pen-operative training with proprietary software merging and rendering simultaneous realities without latency, offering meaningful collaboration and origin views whereby augmented reality merges with and collaborates naturally with remote users.

6. The system of claim 5, whereby multiple communication signals are integrated, minimal physical footprint, natural interactive collaboration is enhanced as remote users' views are not origin user dependent.

7. The system of claim 6, which delivers benefits comprising, in combination:
gamification of virtual/augmented cross-reality merges driven by lack of latency;
incorporation of any needed off of the shelf tools and devices subject to account-based customization;
Realistic and intuitive interfacing, generating meaningful collaboration;
360 degree views creating realistic perspectives and visibility;
Simplified assembly for rapid access;
Hazard reduction and expense via remote personnel support and logistical separation.

8. The system of claim 7, able to be shipped within a single container, requiring no permanent hardware mounts, or other fixed capital intensive equipment to be pre-established, prior to full operation.

9. The system of claim 8, further comprising software to at least:
compose needed visual and acoustic data sets to support training, proctoring and medical diagnostic, and interventional procedures by registering signals from remote sources which enables real-time access to patient data and interaction with these data sets to deliver information, care and diagnostic information to patients, clinicians and students.

10. The system of claim 9, wherein said software enables procedure specific compilations of data structures, mediated by humans and facultively filtered by AI as needed, to optimize outcomes and patient/teachings and training benefits.

11. An open and go system for managing at least one of training, proctoring and procedure support, further comprising, in combination:
at least a container for housing a plurality of omnidirectional cameras and acoustical sensors and transmitters;
customizable data streams for registering and composing images for medical device-based diagnosis, demonstration and procedure support; and,
remote connectivity without latency, whereby real-time medical, interventional and procedural support options may be arrayed, sampled and combined to support optimized outcomes.

12. The open and go system of claim 11, comprising, at least:
an Alienware Gaming Computer;
an Axis PTZ Camera;
a Router; and
ENVISIMED type of Software for registering and composing visual and acoustical images and waveforms.

13. An improved telemedicine platform being fully gamified, portable and able to be rapidly deployed, which comprises, in combination:
a fully equipped procedure room having real-time connections to relevant medical imaging and theatre specific omnidirectional three-hundred and sixty degree views;
HIPAA compliant patient data via proprietary software;
remote clinician and physician access, and,
registered, synchronized and composed aggregates of medical device signals used to compose salient data sets, being software which coordinates lag-free comprehensive access to all patient, provider and equipment driven information, mediated by humans, and supervised by gated AI.
